(19) United States
(12) Patent Application Publication
(10) Pub. No.: US 2013/0150696 A1
(43) Pub. Date: Jun. 13, 2013

(54) HYBRID MULTIELECTRODE ARRAYS

(71) Applicant: Huntington Medical Research Institutes, Pasadena, CA (US)

(72) Inventors: Martin Han, Pasadena, CA (US); Douglas B. McCreery, Altadena, CA (US)

(73) Assignee: HUNTINGTON MEDICAL RESEARCH INSTITUTES, Pasadena, CA (US)

(21) Appl. No.: 13/712,927

(22) Filed: Dec. 12, 2012

Related U.S. Application Data
(60) Provisional application No. 61/569,721, filed on Dec. 12, 2011.

(51) Int. Cl. A61B 5/0478; A61N 1/05

(52) U.S. Cl. CPC A61B 5/0478; A61N 1/0534
USPC 600/378; 29/825

(57) ABSTRACT

A method of forming a multielectrode array includes forming an alignment plate having at least two openings, forming an array bottom plate having at least two openings corresponding to the at least two openings of the alignment plate, temporarily fixing the array bottom plate to the alignment plate, inserting one or more probes or array sub-assemblies into the at least two openings, fixing the probes or array sub-assemblies to the array bottom plate, and removing the array bottom plate from the alignment plate to form a multielectrode array.

### CROSS-REFERENCE TO RELATED APPLICATION

This application claims priority to and the benefit of U.S. Provisional Application No. 61/569,721, filed Dec. 12, 2011, the entire contents of which are incorporated herein by reference.

### FIELD OF THE INVENTION

The present invention is related to a microelectrode array for a brain-machine interface and neural recording and stimulation, and a method of making the same.

### BACKGROUND

Microelectrodes may be implanted into a brain in order to monitor neurological signals and/or deliver therapy to the patient's brain. Deep brain electrodes may be used to investigate and treat a variety of neurological conditions. Electrodes intended to penetrate into neural tissue may have a single recording or stimulating site or multiple sites. As used herein, these electrodes are called single-shank probes, or simply probes. An array can include a plurality of probes, each probe of the array having a single recording or stimulating site or multiple recording or stimulating sites.

Differences in probe packaging, interconnections, and surgical methods make it difficult to directly compare electrode performance in experimental studies, making it difficult to improve the brain-machine interface. For example, the ability of probes implanted chronically in the brain to record resolvable neuronal activities is often reduced or completely lost over time. However, the efficacy and rate of degradation of different types of probes may be difficult to compare objectively when they are not contained in a single array.

Furthermore, it may also be beneficial to combine different types of probes into a single array so that the benefits of several different types of probes may be realized in a single array. However, it is difficult to combine multiple types of probes into a single array. This is due to the small size of the probes and the difficulty in precisely aligning the small probes and in keeping lead wires safe during the assembly. In addition, bonding of lead wires to multisite silicon-based probes is quite different from microwire-based electrodes, or multi-electrode modules supplied by commercial vendors.

Accordingly, there is a need for a method to combine multiple probes and multiple types of probes into a single hybrid microelectrode array.

### SUMMARY

Aspects of the present invention are directed to a multielectrode array that includes multiple probes or array sub-assemblies, and a method of making the same.

According to one embodiment, a method of forming a multielectrode array includes forming an array bottom plate, the array bottom plate having a first opening, a second opening, a first alignment opening, and a second alignment opening. The method further includes forming an alignment plate, the alignment plate having a third opening and a fourth opening corresponding to the first opening and the second opening, respectively, a third alignment opening and a fourth alignment opening corresponding to the first alignment opening and the second alignment opening, respectively. An anti-wicking plate may be applied to the alignment plate. A first alignment member may be placed through the first alignment opening and the third alignment opening and a second alignment member may be placed through the second alignment opening and the fourth alignment opening to align the alignment plate and the array bottom plate. The array bottom plate may be temporarily affixed to the anti-wicking plate. A first probe or array sub-assembly may be inserted into the first opening and the third opening and a second probe or array sub-assembly may be inserted into the second opening and the fourth opening so that at least a portion of each of the first probe or array sub-assembly and the second probe or array sub-assembly extends above the array bottom plate. Each of the first probe or array sub-assembly and the second probe or array sub-assembly may be fixed to the array bottom plate. Then, the array bottom plate with the first probe or array sub-assembly and the second probe or array sub-assembly may be removed from the alignment plate to form a multielectrode array.

The array bottom plate may be adhered to the anti-wicking plate using a temporary adhesive.

The applying the anti-wicking plate to the alignment plate may include forming the anti-wicking plate and fixing the anti-wicking plate to the alignment plate using a permanent adhesive. Or, the applying the anti-wicking plate to the alignment plate may include integrally forming the anti-wicking plate and the alignment plate using 3-D printing.

The method may further include applying an edge strip to a surface of the array bottom plate opposite the anti-wicking plate. The applying the edge strip to a surface of the array bottom plate may include forming the edge strip and fixing the edge strip to the array bottom plate using a permanent adhesive. A permanent adhesive may be applied on top of the array bottom plate up to the edge strip and then cured to secure the first probe or array sub-assembly and the second probe or array sub-assembly in place. The adhesive may partially cured prior to being applied on top of the array bottom plate. The edge strip may be affixed to the array bottom plate using a first permanent, biocompatible adhesive, and a second permanent, biocompatible adhesive may be applied on the array bottom plate up to the edge strip and then cured to form an adhesive cap.

The alignment member may be a wire.

The first probe or array sub-assembly may be different from the second probe or array sub-assembly. The first probe or array sub-assembly may have a different shape than the second probe or array sub-assembly.

The forming the alignment plate may include forming two alignment plates each having the third opening, the fourth opening, the third alignment opening, and the fourth alignment opening corresponding to the first opening, the second opening, the first alignment opening, and the second alignment opening, respectively, wherein a first alignment tube is located in the first opening and the third opening between the two alignment plates, and a second alignment tube is located in the second opening and the fourth opening between the two alignment plates.

The array bottom plate, the alignment plate, and the anti-wicking plate may each include a material independently selected from silicon, a ceramic, a metal, an alloy, and a polymer. In some embodiments, each of the array bottom plate, the alignment plate, and the anti-wicking plate may be made of silicon.

The forming the array bottom plate may include using photolithography and micromachining to form the array bottom plate and the forming the alignment plate may include using photolithography and micromachining to form the array bottom plate.

According to one embodiment, a multielectrode array may include an array bottom plate, a first probe or array sub-assembly and a second probe or array sub-assembly extending below the array bottom plate, the first probe or array sub-assembly and the second probe or array sub-assembly being different, and an adhesive cap above the array bottom plate, the adhesive cap fixing the first probe or array sub-assembly and the second probe or array sub-assembly to the array bottom plate.

The first probe or array sub-assembly may have a different shape than the second probe or array sub-assembly.

The array bottom plate may include a first opening through which the first probe or array sub-assembly extends and a second opening through which the second probe or array sub-assembly extends through the second opening.

The array bottom plate may include a material selected from silicon, a ceramic, a metal, an alloy, and a polymer.

### BRIEF DESCRIPTION OF THE DRAWINGS

FIG. 1A is a schematic perspective view of an apparatus for assembling a hybrid array according to one embodiment.

FIG. 1B is a cross-sectional view of the apparatus of FIG. 1A.

FIG. 2 is a view of an alignment plate according to one embodiment.

FIG. 3 is a view of a support between two alignment plates according to one embodiment.

FIG. 4 is a view of an anti-wicking plate according to one embodiment.

FIG. 5 is a view of a bottom plate according to one embodiment.

FIG. 6 is a view of an edge strip according to one embodiment.

FIG. 7 is a view of a multielectrode array according to one embodiment.

### DETAILED DESCRIPTION

According to some embodiments, a method of forming a multielectrode array including at least two different electrodes includes forming an apparatus for assembling an array, assembling an array, and removing the array from the assembly.

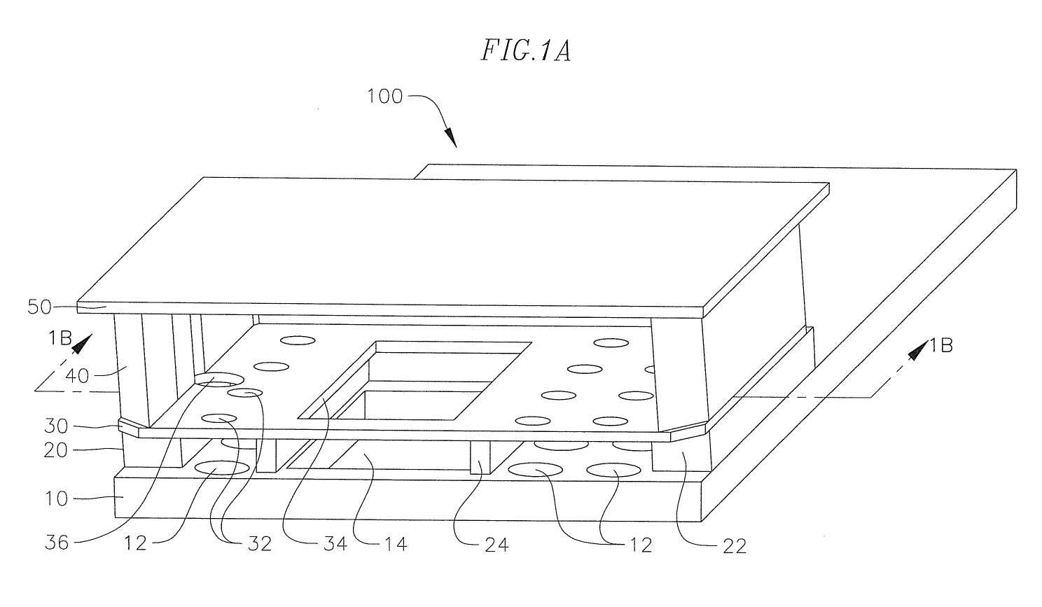

FIG. 1A is a schematic perspective view of the apparatus 100 for assembling an array, and FIG. 1B is a cross-sectional view of the apparatus of FIG. 1A with the cover plate 50 omitted. As shown in FIGS. 1A and 1B, the apparatus 100 for assembling an array includes at least one alignment plate 10, an anti-wicking plate 20, an array bottom plate 30, an array edge strip 40, and an optional array cover plate 50 (omitted in FIG. 1B). Each of these components may be made of any suitable material, keeping in mind the small size of the array and probes and the durability required for the array and the apparatus. In some embodiments, the components may be made of silicon, a metal or alloy, a ceramic, or a polymer (e.g., a hard polymer). When the components are made of silicon, they may be formed by photolithography and micromachining (e.g., using deep reactive ion etching) on a silicon wafer. However, any suitable method of patterning the substrate material may be used, for example, etching, or laser micromachining. When the components are made of a polymer, they may be formed by filling a mold with the polymer, or alternatively, the components may be made by other manufacturing methods such as injection molding, calendaring, printing, or the like. Each component may be formed of the same material by the same process, or different materials and processes may be used. When the components are manufactured by 3-D printing or micromachining of silicon wafers (or other similar bulk-etching technology), multiple plates could be integrated together rather than forming them separately and adhering them to one another. For example, the edge plate could be integrated with the array bottom plate and the anti-wicking plate could be integrated with the alignment plate. The components may preferably be made of silicon using photolithography on a silicon wafer.

The apparatus 100 is made specific to a specific array configuration. Thus, the size and number of openings in the various components may be adjusted according to the desired array. For example, as shown in FIG. 1A, the apparatus may be designed to accommodate 12 individual probes and a module of 8 electrodes (e.g., a 4×2 Utah Intracortical Array from Blackrock Microsystems, Inc., hereinafter called a “Blackrock array”) (hereinafter, an array of electrodes is generally called an “array sub-assembly”) fabricated by one or more commercial vendors. As such, in FIG. 1A, there are 12 circular probe openings 32 and one rectangular opening 34 in the bottom plate 30 and 12 circular probe openings 12 and one rectangular opening 14 in the alignment plate 10. However, any number of openings may be formed in the bottom plate 30 and the alignment plate 10. Furthermore, the size and shape of the openings may be varied according to the type of electrode used. For example, a hybrid array including a Blackrock 4×2 array may include a rectangular opening 34 in the bottom plate 30 and a rectangular opening 14 in the alignment plate 10. Similarly, the probe openings may be circular to correspond to a generally cylindrical probe or any other probe that would fit and be aligned by the circular shape of the openings. The probe openings may also be smaller rectangular shapes to fit single probes that are rectangular prisms, such as a multi-site probe fabricated by a commercial vendor. In addition, the layout of the openings, and thus, the configuration of the resulting hybrid array, may be varied as desired. For example, while the embodiment shown in FIG. 1A is a generally rectangular layout, the probes could be laid out in a circular pattern to form a circular hybrid array. Alternatively, any suitable layout could be used.

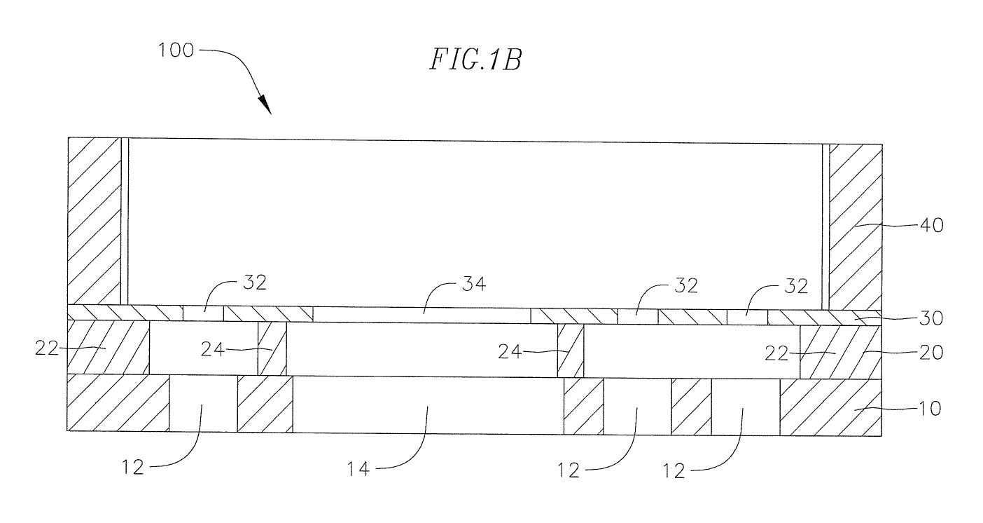

FIG. 2 depicts an alignment plate according to an embodiment. First, at least one alignment plate 10 is formed as shown in FIG. 2. As stated above, the alignment plate includes one opening for each probe or array sub-assembly. In FIG. 2, the alignment plate 10 includes 12 circular probe openings 12, one rectangular opening 14, and two alignment openings 16. As stated above, the number of openings, shape of the openings, and arrangement of the openings may be varied according to the desired hybrid array. As shown in FIGS. 1A and 2, the alignment plate 10 may have an elongated end 19. The elongated end 19 may allow the alignment plate to be fixed to a flat object to assist in the alignment of the apparatus 100. The alignment plate 10 may also have a notch 18 to indicate where the remainder of the components should be fixed to the alignment plate 10. The notch 18 assists in alignment of the remainder of the components, and may have any suitable shape, and may match the notches or chamfers in the other components. In one embodiment, the alignment plate may be 3 mm wide and 6 mm long. The probe openings 12 may have a diameter of 0.17 mm, which could be the diameter of the probes. Alternatively, the probe openings 12 may be slightly larger than the diameter of the probes. In yet another embodiment, described below, the probe openings 12 may be larger, e.g., 0.27 mm, to allow for a stainless steel alignment tube to be inserted therein. The opening for the Blackrock 4×2 array may be 1.0 mm wide by 1.5 mm long, which is larger than the area of the Blackrock array. As the Blackrock 4×2 array is aligned by being seated on the surface of the bottom plate 30, the array may not need further alignment by the alignment plate 10. However, the alignment plate could be adjusted to align an array as necessary. The circular alignment openings 16 may have a diameter of 0.26 mm. However, the alignment openings 16 may have any suitable shape and diameter to allow an alignment wire to be inserted therein to align the components. The notch may be at a distance from the end that corresponds to the length of the bottom plate 30, e.g., 2.2 mm. The alignment plate may be about 200 μm thick (e.g., 200 μm). However, the alignment plate may have any suitable thickness.

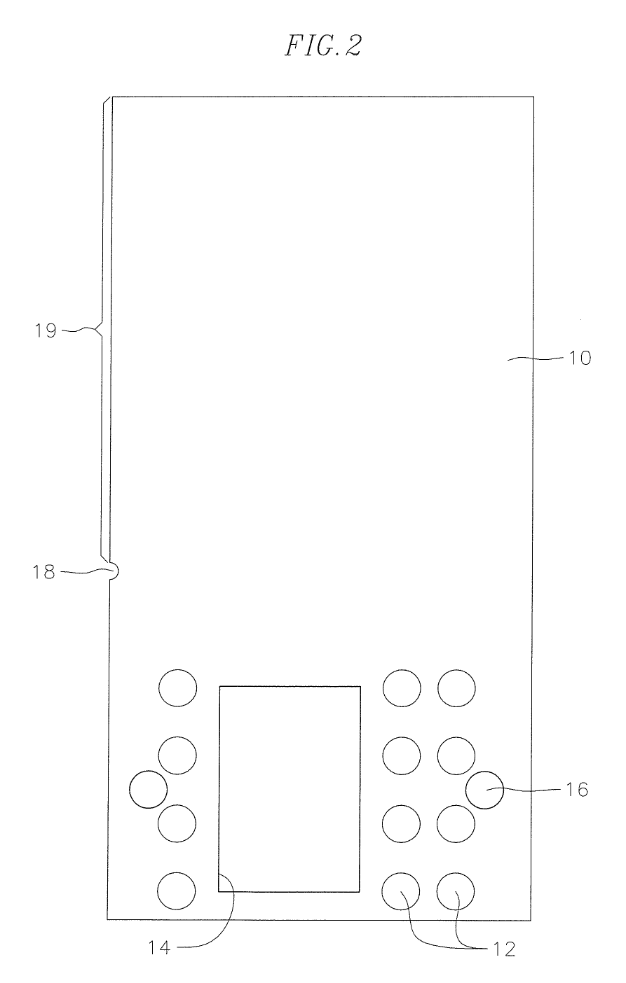

FIG. 3 depicts a pair of alignment plates fixed to a support and a plurality of tubes between the alignment plates. As shown in FIG. 3, two alignment plates 10 may be used, and stainless steel tubes 11 may be inserted into the openings 12 and extend between the two alignment plates 10. The two alignment plates 10 may be the same, as shown in FIG. 3, or alternatively, the two alignment plates may have different sizes. The stainless steel tubes 11 may be fixed to the alignment plates using a polymer, epoxy, or any other suitable adhesive. Each stainless steel tube 11 may have an inner diameter that is slightly wider than the largest diameter or width of the probe that is to be inserted therein. Stainless steel tubes 11 may also extend between the alignment openings 16. The distance between the two alignment plates 10 may be sufficient to allow the longest probe to extend within its stainless steel tube. Alternatively, the stainless steel tubes 11 may extend beyond the bottom of the bottom alignment plate. Or in some embodiments, the stainless steel tubes 11 may only extend between the two alignment plates, and the probes to be inserted therein may extend beyond the stainless steel tubes. The optional second alignment plate and tubes aid in aligning the probes.

In some embodiments, the top alignment plate may be fixed to the top of a support 60, as shown in FIG. 3, using an epoxy or any suitable adhesive. The support 60 may be any rigid material, such as metal. The support 60 may be used to fix the assembly during manufacture of the array. For example, one or more bolts or other fasteners may fix the support 60 to a work surface so that the alignment plates 10, and thus the apparatus 100 is firmly secured during manufacture of the hybrid array. In some embodiments, the bottom alignment plate 10, the stainless steel tubes, and/or the support 60 are omitted. For example, when a probe is a rectangular prism, the associated opening in the alignment plate 10 may be rectangular and may be just larger than the width of the probe, and thus, the rectangular opening of the bottom plate 30 and a single alignment plate 10 may align the probe. In such an instance, no lower alignment plate or stainless steel tubes are necessary. Alternatively, the two alignment plates may be used without stainless steel tubes. That is, the rectangular prism probe may be aligned by the two openings of the two alignment plates. In addition, a separate support 60 may not be necessary as long as the assembly is fixed during manufacture.

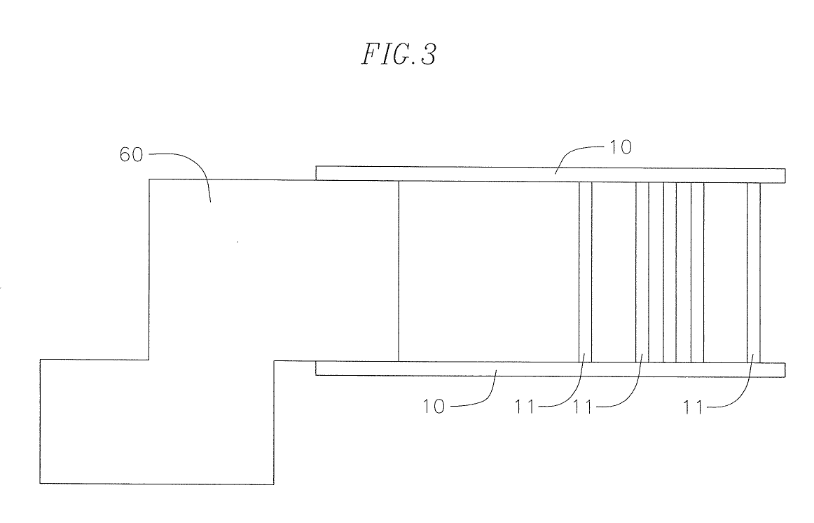

The apparatus may include an anti-wicking plate as shown in FIGS. 1A, 1B, and 4. An anti-wicking plate 20 may be fixed to the top surface of the alignment plate 10. The anti-wicking plate 20 prevents uncured polymer or epoxy used to fix each probe or array to the bottom plate 30 (described below) from wicking between the bottom plate 30 and the alignment plate 10 and/or the stainless steel tubes 11. If the uncured polymer or epoxy is allowed to wick by capillary action along closely approximated surfaces between the bottom plate 30 and the alignment plate 10 or the stainless steel tubes 11, the bottom plate 30 and the alignment plate 10 or stainless steel tubes 11 may be permanently bonded together. The anti-wicking plate may also act as a spacer between the bottom plate 30 and the alignment plate 10. In one embodiment, the anti-wicking plate 20 includes two outer arms 22 and two inner arms 24. The two outer arms include a cut-out 26 for the alignment member, discussed later. The anti-wicking plate 20 may be open at one end. The other end may be a back region 28. A space may be between each of the outer arms 22 and the adjacent inner arms 24 for the plurality of individual probes. The space between the two inner arms 24 may be for a module of probes (e.g., an array sub-assembly). However, more or fewer arms may be included in the anti-wicking plate, as needed to provide sufficient stability for the bottom plate, depending on the particular hybrid array. FIG. 4 includes phantom lines to show the location of the openings of alignment plate 10 or the bottom plate 30. The arms 22 and 24 should be sufficiently spaced from each probe or array so that uncured polymer does not wick down to the alignment plate or the stainless steel tubes 11. For example in one embodiment, the anti-wicking plate 20 may have an overall width of 3 mm and a length of 2 mm (which is a smaller length than the length of the bottom plate). The outer arms 22 may have a width of 0.3 mm (except for the cut-out 26 for the alignment wire), and the inner arms may have a width of 0.1 mm. As such, the space between the left outer arm 22 and the left inner arm 24 may be about 0.4 mm, which provides sufficient space to prevent wicking. The back region may be 0.3 mm deep. The anti-wicking plate 20 may be about 200 μm thick (e.g., 200 μm thick). However, any suitable widths and lengths for the arms and the back region may be used, as necessary, for a given hybrid array.

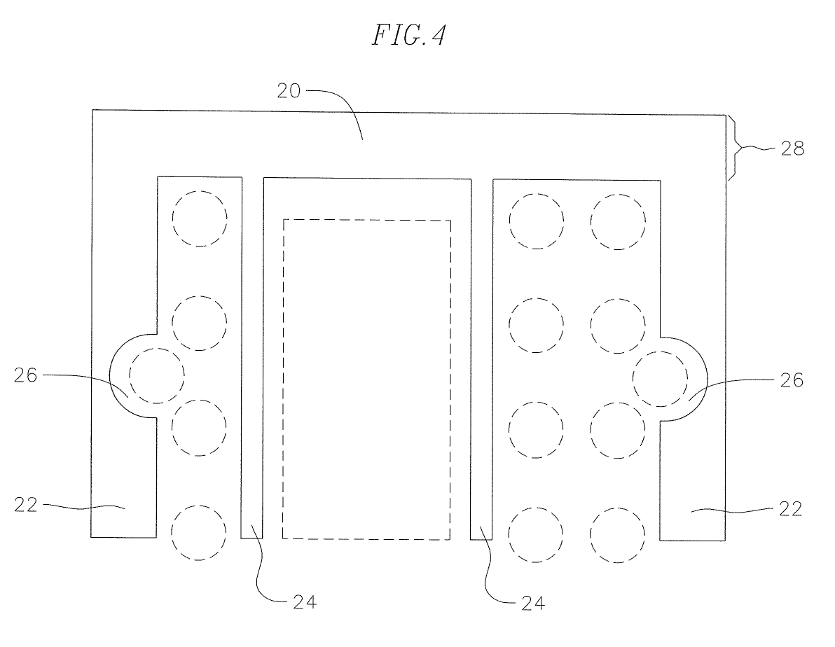

FIG. 5 depicts a bottom plate according to an embodiment. A bottom plate 30 may be temporarily affixed to the top surface of the anti-wicking plate 20. The bottom plate 30 becomes a permanent part of the hybrid array. In FIG. 5, the bottom plate 30 includes 12 circular probe openings 32, one rectangular opening 34, and two alignment openings 36. As stated above, the number of openings (e.g., one for each probe or array) may be varied according to the desired hybrid array. The openings 32, 34, and 36 correspond to the openings 12, 14, and 16 in the alignment plate 10. Each corner of the bottom plate 30 may be a chamfer. The chamfers 38 may create a smaller footprint for the hybrid array and may also aid in aligning the components that remain with the array. In one embodiment, the bottom plate 30 may be 3.0 mm wide and 2.2 mm long. The probe openings 32 may have a diameter of 0.17 mm, which could be the diameter of the probes to be inserted therein. The opening 34 for the Blackrock 4×2 array may be 0.8 mm wide by 1.5 mm long, which is smaller than the area of the base of the Blackrock 4×2 array. The multi-probe module is aligned by registering with the surface of the bottom plate 30 (i.e., being seated on the surface of the bottom plate). However, the openings 32 and 34 may have any suitable size and shape to fit the probes or arrays inserted therein. The circular alignment openings 36 may be the same or about the same diameter as the alignment openings 16 of the alignment plate 10, e.g., 0.26 mm. Or, the alignment openings 36 may be any other suitable shape that matches the shape of the alignment openings 16 of the alignment plate 10. The bottom plate may be about 50 μm thick (e.g., 50 μm). However, the bottom plate may have any suitable thickness. In some embodiments, the bottom plate may be curved to correspond to the shape of its intended use. For example, if the hybrid array is designed to be used for a human brain, the bottom plate may be curved to correspond to the curved shape of the brain, or if the hybrid array is designed to be used for a spinal cord, the bottom plate may be curved to correspond to the spinal cord. While not described herein, other parts of the assembly (e.g., edge strip, top plate) may each be curved to correspond to the curve of the bottom plate. Or, in some embodiments, some portions of the assembly and/or array may be curved, while other portions are not.

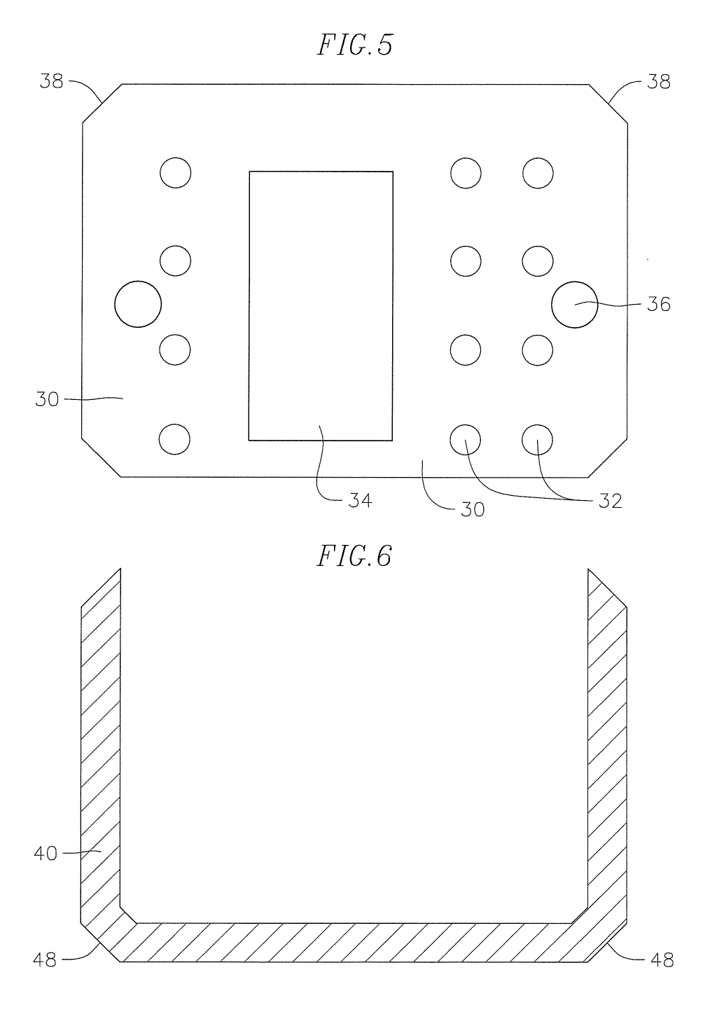

FIG. 6 depicts an edge strip according to an embodiment. An edge strip 40 may be affixed to the top surface of the bottom plate 30. The edge strip 40 and bottom plate together form a cavity to fill with a polymer, epoxy, or other adhesive. That is, the edge strip 40 provides a boundary within which a polymer, epoxy, or other high-viscosity fluid adhesive may be deposited on top of the bottom plate. As shown in FIG. 6, the edge strip 40 may be present on 3 sides. A 3 sided edge strip may be used when the wires connected to the probes and/or arrays exit out one side of the hybrid array. However, in some embodiments, the wires exit out the top, and in such a case, a 4 sided edge strip may be used, which may better contain the fluid polymer. Each of the corners of the edge strip 40 may be chamfered. As with the bottom plate, the chamfers 48 may be used to align the edge strip 40 with the array. The edge strip 40 may be permanently affixed to the top surface of the bottom plate 30, and thus, may become part of the hybrid array. The edge strip may be made to be any suitable height to form a suitable hybrid array body. For example, the edge strip may be about 200 μm to about 800 μm thick (e.g., 200 μm to 800 μm). The edge strip may be a single layer, or multiple thinner layers may be built upon one another to form a deeper cavity. For example, 3 200 μm edge strips may be permanently affixed together to form a 600 μm composite edge strip. However, the edge strip may have any suitable thickness to form any suitable sized array body. The edge strip should have a sufficient height so that the probe tops do not extend above the top of the edge strips.

Last, a cover plate 50 (or top plate) may be attached to the top of the edge strip 40 as shown in FIG. 1A. The cover plate 50 may be used to manipulate the hybrid array after it is formed. For example, a tool may be attached to the top of the cover plate 50 to lift the hybrid array from the anti-wicking plate and the assembly apparatus without damaging the probes in the hybrid array. In some embodiments, the cover plate 50 may be affixed to the top of the edge strip and the cured epoxy using a permanent adhesive, and thus, may become a permanent party of the hybrid array. The cover plate 50 may be a relatively large flat piece that has a greater area than the bottom plate. For example, the cover plate 50 may be about 3.2 mm wide and 2.4 mm long, or any suitable dimensions. In some embodiments, the cover plate 50 may have the same relative dimensions (overall shape and size) as the bottom plate 30 except that it does not include openings. The top plate may be about 50 μm thick (e.g. 50 μm), but it may be any suitable thickness.

Once the components (e.g., alignment plate, anti-wicking spacer, bottom plate, edge strip, top plate) of the apparatus are fabricated, they may be assembled. Those of ordinary skill in the art would understand that the apparatus used to assemble the hybrid array may be fabricated in any suitable order. First, the alignment plate 10 may be fixed to the support 60. Optionally, two alignment plates and/or the stainless steel tubes 11 may be used and assembled, as discussed above. That is, optionally, one alignment plate may be fixed to the top of the support and one alignment plate may be fixed to the bottom of the support, and stainless steel tubes may be inserted into the openings of each alignment plate and fixed thereto. Then, the anti-wicking plate 20 may be permanently fixed to the top surface of the alignment plate 10 using an epoxy, polymer, or other adhesive, and for example, an Epotek epoxy may be used. The alignment plate 10 and the support may then be placed in an oven to allow the adhesive to cure. For example, they may be placed in a low temperature oven for about 15 minutes. Then, the anti-wicking plate 20 may be permanently fixed to the alignment plate 10 using an epoxy, polymer, or other adhesive, and for example, an Epotek epoxy may be used. The anti-wicking plate 20, the alignment plate 10, and the support 60 may be placed in an oven to allow the adhesive to cure, for example, they may be placed in a low temperature oven for about 15 minutes. In fixing the anti-wicking plate 20 to the alignment plate 10, it is important that the edge is aligned, so that the openings in the anti-wicking plate 20 are registered (e.g., lined up) with the openings of the alignment plate. Alternatively, the anti-wicking plate 20 may be fixed to the alignment plate 10 prior to fixing the alignment plate 10 to the support 60.

The edge strip 40 may be attached to the bottom plate 30. The bottom plate 30 may be aligned along two walls (for example, pushed up against two walls of a structure), and then the edge strip 40 may be placed on the bottom plate 30, with a layer of permanent adhesive therebetween. The permanent adhesive may be a polymer, epoxy, or other permanent adhesive, and for example, could be a biocompatible epoxy. The adhesive should be biocompatible because the edge strip and bottom plate are a permanent part of the hybrid array. The bottom plate 30 and the edge strip 40 may then be placed in an oven to allow the adhesive to cure. For example, they may be placed in a low temperature oven for about 15 minutes to cure. Alternatively, the edge strip 40 may be fixed to the bottom plate after the probes and/or arrays are fixed to the bottom plate.

Next, the bottom plate 30 may be attached to the top surface of the anti-wicking plate 20. First, an alignment member (e.g., a thin wire) that fits inside the alignment openings 16 and 36 is placed in one set of the alignment openings. Then, the bottom plate 30 is moved (e.g., rotated around the inserted thin wire) until another thin wire may be inserted into the other alignment opening. 16 and 36. Once aligned, and once the bottom plate 30 is seated firmly on the anti-wicking plate 20, a temporary adhesive may be applied to the junction between the bottom plate 30 and the anti-wicking plate 20. In some embodiments, the temporary adhesive may only be applied along the back surface of the structures (i.e., the widest part of the anti-wicking plate). The temporary adhesive may be a water-soluble adhesive such as polyvinyl alcohol or any other suitable temporary adhesive. The temporary adhesive may be a type such that when the assembly of the hybrid array is completed, a solvent, e.g., water, may be applied to the temporary adhesive to release the bond between the bottom plate 30 and the anti-wicking plate 20 without harming the hybrid array.

Then, the probes and/or array sub-assemblies may then be inserted into the apparatus 100. In view of the fragile nature of the probes, care must be taken when inserting probes into the apparatus. As such, in some embodiments, a micromanipulator or other suitable device for intricate manipulation may be used. However, the probes may be inserted by any suitable method. Each probe may then be inserted into a probe opening 32 (and accordingly, probe opening 12) using the micromanipulator. The opening 12 in the alignment plate aligns the axis of the probes. The amount of extension of each probe below the array bottom plate may be controlled by a protuberance on the probe that is greater in size than the diameter of the opening 32 (e.g., in the case of a NeuroNexus probe, its bonding zone or area which contains bond pads). The array sub-assembly including a plurality of probes (e.g., a Blackrock 4×2) is seated in the array sub-assembly opening 34, and should be seated so that the electrodes of the array sub-assembly are approximately an equal distance from the front and back and left and right edges of the opening 36. Once all probes and array sub-assemblies have been inserted, and they have been seated against the bottom plate 30, a very small amount of an adhesive is applied where each probe or array contacts the bottom plate 30. For example, a fast-curing epoxy or other permanent adhesive that is biocompatible may be used. Alternatively, the probes may be fixed one at a time, as they are inserted, or, once a group of probes (e.g., a row) is inserted, they may be fixed. In some embodiments, an electrode or array is not inserted into one or more openings. That is, the bottom plate and/or alignment plate may include unused openings.

The lead wires may be any suitable lead wires, such as those commonly used with penetratable electrode probes. For example, gold lead wires may be used. In addition, gold lead wires coated with Parylene-C may be used.

Then the lead wires are directed out of the structure and attached to a connector. In some embodiments, when the top plate is going to be used, the lead wires are directed out the open side of the structure (where the edge strip is not present). In other embodiments, when a top plate is not going to be used, the lead wires may be extended vertically out of the structure (i.e., in a direction generally perpendicular to the surface of the bottom plate). Then, the secured probes may be further secured by applying partly cured epoxy or other adhesive along the seated and fixed row. The epoxy or other adhesive is partly cured to increase its viscosity and prevent the adhesive from seeping through the openings or spreading along the array bottom. Alternatively, once a single probe or a group of probes (e.g., a row) is inserted, they may be further secured by applying an epoxy or other adhesive that is partly cured. The direction of the lead wire can restrict or enable the hybrid array's use for a specific purpose. For example, if the lead wires exit through the area where the top of the hybrid array, the hybrid array can be used with deep brain structures. On the other hand, if the leads exit through the side of the hybrid array, the hybrid array can be used for the top of the brain or the cerebral cortex.

Once all probes and/or array sub-assemblies have been seated and fixed to the bottom plate 30, the cavity is filled with a polymer, epoxy, or other adhesive. For example, Epotek 301 may be used. The adhesive may be partially cured when it is applied to the cavity so that it does not flow beyond the cavity. For example, when Epotek 301 is used, it may be allowed to partly polymerize for about 2.5 hours at room temperature. The partial curing of the Epotek 301 may be tested by lifting a drop with 0.001″ wire; when it requires about 3 seconds for the string to retract into the drop on the end of the wire, an appropriate amount of curing has occurred for use. Slowly, the cavity may be filled with adhesive. The adhesive should not extend above the top of the edge strip 40. This forms the body of the hybrid array.

The cover plate 50 may be fixed to the top of the edge strip. The cover plate may also be in contact with the adhesive filling the cavity. When the adhesive is fully cured, a lifting surface controlled by a micromanipulator is joined to the upper plate using a water-soluble adhesive. Water or another solvent is applied to the junction between the array bottom plate and the anti-wicking plate to break the adhesive bond and separate the bottom plate and the anti-wicking plate, and the array is lifted from the anti-wicking plate using the micromanipulator. The micromanipulator may then be used to lower the structure into a solvent to release the array from the micromanipulator. In some embodiments, no cover plate is used.

Any suitable penetratable electrode probes and/or array sub-assemblies may be used. In some embodiments, the probes may be made of silicon, for example, boron-doped silicon devices from NeuroNexus Inc. or Utah Intracortical Arrays from Blackrock Microsystems, Inc., thick silicon probes shaped by deep reactive ion etching, or the like; carbon; metal, for example, iridium, platinum, gold, alloys such as a platinum-iridium alloy, or the like; diamond; compound semiconductor materials, for example GaAs, InP, or the like; polymers for example, polyimide, Parylene, silicone/PDMS, or the like; or biodegradable polymers, for example, poly-lactic-glycolic acid or the like. The type of probes may be single-site electrodes, for example, metal-based microwires or the like; multi-site electrodes, for example, silicon devices, polymer devices, or the like; or metal microwires with open site(s) along the length of the wire; or the like. However, any suitable probe materials and any suitable types of probes may be used.

As used herein, “polymer, epoxy, or other adhesive” generally refers to any material that adheres or bonds two things together. The polymer, epoxy, or other adhesive may include a filler. As used herein, a temporary adhesive refers to an adhesive, such as a polymer or an epoxy, that is dissolved by a common solvent that does not harm the array. For example, an adhesive such as polyvinyl alcohol that dissolves with water is a temporary adhesive. As used herein, a permanent adhesive refers to an adhesive, such as a polymer or epoxy, commonly known as a permanent adhesive. For example, a permanent adhesive may be an adhesive that is not dissolved by a solvent that does not harm the array or is only dissolved by an uncommon solvent.

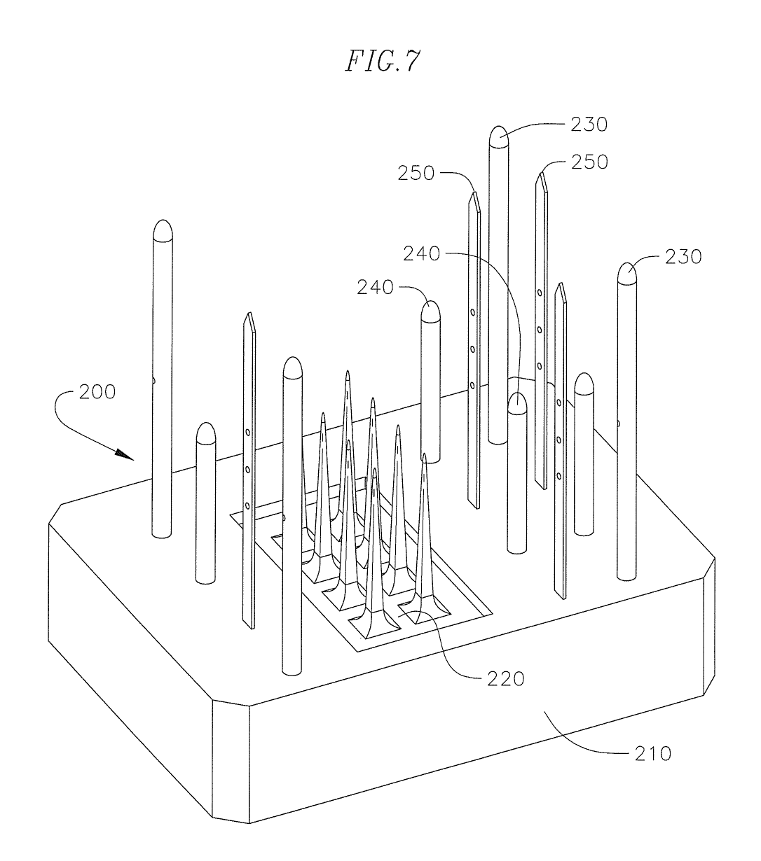

Using this method, a hybrid multi-electrode array may be formed as shown in FIG. 7. As discussed above, the hybrid array may be formed to have any suitable number of probes and/or array sub-assemblies as desired. In FIG. 7, the hybrid multi-electrode array 200 includes an array base 210, a plurality of different probes 230, 240, and 250, and an array sub-assembly 220. While not shown in detail in FIG. 7, the base 210 may include a bottom plate 30, an edge strip 40, and a cover plate 50. While some previous electrode arrays have been able to combine multiple similar electrodes (e.g., a Blackrock module), the present method allows the formation of an array that includes multiple dissimilar electrodes. By integrating multiple commercially available probes or array sub-assemblies into a single hybrid array, the probes or array sub-assemblies can be compared more objectively in order to compare their performance and thus their suitability for particular applications in experimental neuroscience and clinical applications. Or, by integrating multiple commercially available probes or array sub-assemblies, the benefits of combining multiple types of probes or array sub-assemblies in the same hybrid array may be achieved. For example, by arranging single-tip electrodes and multisite probes in the same hybrid array, one can record and stimulate from one type to another, or in any combination, according to the brain circuitry, for example, in different cortical lamina.

The preceding description has been presented with reference to certain exemplary embodiments of the invention. Workers skilled in the art and technology to which this invention pertains will appreciate that alterations and changes to the described embodiments may be practiced without meaningfully departing from the spirit and scope of this invention, as defined in the appended claims. It is further understood that the drawings are not necessarily to scale.

Accordingly, the foregoing description should not be read as pertaining only to the precise structures and methods described and illustrated in the accompanying drawings, but rather should be read consistent with and as support to the following claims which are to have their fullest and fairest scope.

What is claimed is:

1. A method of forming a multielectrode array, the method comprising:
forming an array bottom plate, the array bottom plate having a first opening, a second opening, a first alignment opening, and a second alignment opening;
forming an alignment plate, the alignment plate having a third opening and a fourth opening corresponding to the first opening and the second opening, respectively, a third alignment opening and a fourth alignment opening corresponding to the first alignment opening and the second alignment opening, respectively;
applying an anti-wicking plate to the alignment plate;
placing a first alignment member through the first alignment opening and the third alignment opening and placing a second alignment member through the second alignment opening and the fourth alignment opening to align the alignment plate and the array bottom plate;
temporarily affixing the array bottom plate to the anti-wicking plate;
inserting a first probe or array sub-assembly into the first opening and the third opening and inserting a second probe or array sub-assembly into the second opening and the fourth opening so that at least a portion of each of the first probe or array sub-assembly and the second probe or array sub-assembly extend above the array bottom plate;
fixing each of the first probe or array sub-assembly and the second probe or array sub-assembly to the array bottom plate; and
removing the array bottom plate with the first probe or array sub-assembly and the second probe or array sub-assembly from the alignment plate to form a multielectrode array.

2. The method of forming a microelectrode array of claim 1, wherein the array bottom plate is adhered to the anti-wicking plate using a temporary adhesive.

3. The method of forming a microelectrode array of claim 1, wherein the applying the anti-wicking plate to the alignment plate comprises forming the anti-wicking plate and fixing the anti-wicking plate to the alignment plate using a permanent adhesive.

4. The method of forming a microelectrode array of claim 1, wherein the applying the anti-wicking plate to the alignment plate comprises integrally forming the anti-wicking plate and the alignment plate using 3-D printing.

5. The method of forming a microelectrode array of claim 1, the method further comprising applying an edge strip to a surface of the array bottom plate opposite the anti-wicking plate.

6. The method of forming a microelectrode array of claim 5, wherein the applying the edge strip to a surface of the array bottom plate comprises forming the edge strip and fixing the edge strip to the array bottom plate using a permanent adhesive.

7. The method of forming a microelectrode array of claim 5, wherein a permanent adhesive is applied on top of the array bottom plate up to the edge strip and then cured to secure the first probe or array sub-assembly and the second probe or array sub-assembly in place.

8. The method of forming a microelectrode array of claim 7, wherein the adhesive is partially cured prior to being applied on top of the array bottom plate.

9. The method of forming a microelectrode array of claim 5, wherein the edge strip is affixed to the array bottom plate using a first permanent, biocompatible adhesive, and wherein a second permanent, biocompatible adhesive is applied on the array bottom plate up to the edge strip and then cured to form an adhesive cap.

10. The method of forming a microelectrode array of claim 1, wherein the alignment member comprises a wire.

11. The method of forming a microelectrode array of claim 1, wherein the first probe or array sub-assembly is different from the second probe or array sub-assembly.

12. The method of forming a microelectrode array of claim 1, wherein the first probe or array sub-assembly has a different shape than the second probe or array sub-assembly.

13. The method of forming a microelectrode array of claim 1, wherein the forming the alignment plate comprises forming two alignment plates each having the third opening, the fourth opening, the third alignment opening, and the fourth alignment opening corresponding to the first opening, the second opening, the first alignment opening, and the second alignment opening, respectively, wherein a first alignment tube is located in the first opening and the third opening between the two alignment plates, and a second alignment tube is located in the second opening and the fourth opening between the two alignment plates.

14. The method of forming a microelectrode array of claim 1, wherein each of the array bottom plate, the alignment plate, and the anti-wicking plate comprise a material independently selected from the group consisting of silicon, a ceramic, a metal, an alloy, and a polymer.

15. The method of forming a microelectrode array of claim 14, wherein each of the array bottom plate, the alignment plate, and the anti-wicking plate comprise silicon.

16. The method of forming a microelectrode array of claim 1, wherein the forming the array bottom plate comprises using photolithography and micromachining to form the array bottom plate and the forming the alignment plate comprises using photolithography and micromachining to form the array bottom plate.

17. A multielectrode array comprising:
an array bottom plate;
a first probe or array sub-assembly and a second probe or array sub-assembly extending below the array bottom plate, the first probe or array sub-assembly and the second probe or array sub-assembly being different; and
an adhesive cap above the array bottom plate, the adhesive cap fixing the first probe or array sub-assembly and the second probe or array sub-assembly to the array bottom plate.

18. The multielectrode array of claim 17, wherein the first probe or array sub-assembly has a different shape than the second probe or array sub-assembly.

19. The multielectrode array of claim 17, wherein the array bottom plate comprises a first opening through which the first probe or array sub-assembly extends and a second opening through which the second probe or array sub-assembly extends through the second opening.

20. The multielectrode array of claim 17, wherein the array bottom plate comprises a material selected from the group consisting of silicon, a ceramic, a metal, an alloy, and a polymer.
